(19) United States
(12) Patent Application Publication
(10) Pub. No.: US 2005/0177315 A1
(43) Pub. Date: Aug. 11, 2005

(54) Feature extraction of partial microarray images

(76) Inventor: Srinka Ghosh, San Francisco, CA (US)

Correspondence Address: AGILENT TECHNOLOGIES, INC.;Legal Department, DL429, Intellectual Property Administration, P.O. Box 7599, Loveland, CO, 80537-0599, US

(21) Appl. No.: 10/773,890

(22) Filed: Feb. 6, 2004

(51) Int. Cl. G06F 19/00; G01N 33/48; G01N 33/50

(52) U.S. Cl. 702/20

(57) ABSTRACT

A microarray processing system provides to a user an ability to draw one or more contour lines around portions of the microarray considered by the user to be undamaged, non-defective, and otherwise not compromised and therefore suitable for feature extraction. The microarray processing system then constructs one or more rectangular regions of feature extractability based on the user-indicated subregions of feature extractability, and proceeds to extract data from the one or more rectangular regions of feature extractability.

### BACKGROUND OF THE INVENTION

The present invention relates to processing of microarray images. In order to facilitate discussion of the present invention, in following sections, a brief description of nucleic-acid-polymer-based microarrays is provided in following paragraphs of the current subsection. Although the method and system of the present invention may be employed to extract data from any type of microarray, including protein-based microarrays and microarrays with natural or synthetic small-molecule, polymer, or macromolecule-based probes targeting any of a wide range of natural or synthetic probe-binding target molecules, nucleic-acid-based microarrays are currently commonly used, and therefore provide a reasonable basis for examples used in following subsections to illustrate the method and system of the present invention.

Array technologies have gained prominence in biological research and are likely to become important and widely used diagnostic tools in the healthcare industry. Currently, microarray techniques are most often used to determine the concentrations of particular nucleic-acid polymers in complex sample solutions. Molecular-array-based analytical techniques are not, however, restricted to analysis of nucleic acid solutions, but may be employed to analyze complex solutions of any type of molecule that can be optically or radiometrically scanned and that can bind with high specificity to complementary molecules synthesized within, or bound to, discrete features on the surface of an array. Because arrays are widely used for analysis of nucleic acid samples, the following background information on arrays is introduced in the context of analysis of nucleic acid solutions following a brief background of nucleic acid chemistry.

Deoxyribonucleic acid (“DNA”) and ribonucleic acid (“RNA”) are linear polymers, each synthesized from four different types of subunit molecules. The subunit molecules for DNA include: (1) deoxy-adenosine, abbreviated “A,” a purine nucleoside; (2) deoxy-thymidine, abbreviated “T,” a pyrimidine nucleoside; (3) deoxy-cytosine, abbreviated “C,” a pyrimidine nucleoside; and (4) deoxy-guanosine, abbreviated “G,” a purine nucleoside. The subunit molecules for RNA include: (1) adenosine, abbreviated “A,” a purine nucleoside; (2) uracil, abbreviated “U,” a pyrimidine nucleoside; (3) cytosine, abbreviated “C,” a pyrimidine nucleoside; and (4) guanosine, abbreviated “G,” a purine nucleoside. FIG. 1 illustrates a short DNA polymer 100, called an oligomer, composed of the following subunits: (1) deoxy-adenosine 102; (2) deoxy-thymidine 104; (3) deoxy-cytosine 106; and (4) deoxy-guanosine 108. When phosphorylated, subunits of DNA and RNA molecules are called “nucleotides” and are linked together through phosphodiester bonds 110-115 to form DNA and RNA polymers. A linear DNA molecule, such as the oligomer shown in FIG. 1, has a 5′ end 118 and a 3′ end 120. A DNA polymer can be chemically characterized by writing, in sequence from the 5′ end to the 3′ end, the single letter abbreviations for the nucleotide subunits that together compose the DNA polymer. For example, the oligomer 100 shown in FIG. 1 can be chemically represented as “ATCG.” A DNA nucleotide comprises a purine or pyrimidine base (e.g. adenine 122 of the deoxy-adenylate nucleotide 102), a deoxy-ribose sugar (e.g. deoxy-ribose 124 of the deoxy-adenylate nucleotide 102), and a phosphate group (e.g. phosphate 126) that links one nucleotide to another nucleotide in the DNA polymer. In RNA polymers, the nucleotides contain ribose sugars rather than deoxy-ribose sugars. In ribose, a hydroxyl group takes the place of the 2′ hydrogen 128 in a DNA nucleotide. RNA polymers contain uridine nucleosides rather than the deoxy-thymidine nucleosides contained in DNA. The pyrimidine base uracil lacks a methyl group (130 in FIG. 1) contained in the pyrimidine base thymine of deoxy-thymidine.

The DNA polymers that contain the organization information for living organisms occur in the nuclei of cells in pairs, forming double-stranded DNA helixes. One polymer of the pair is laid out in a 5′ to 3′ direction, and the other polymer of the pair is laid out in a 3′ to 5′ direction. The two DNA polymers in a double-stranded DNA helix are therefore described as being anti-parallel. The two DNA polymers, or strands, within a double-stranded DNA helix are bound to each other through attractive forces including hydrophobic interactions between stacked purine and pyrimidine bases and hydrogen bonding between purine and pyrimidine bases, the attractive forces emphasized by conformational constraints of DNA polymers. Because of a number of chemical and topographic constraints, double-stranded DNA helices are most stable when deoxy-adenylate subunits of one strand hydrogen bond to deoxy-thymidylate subunits of the other strand, and deoxy-guanylate subunits of one strand hydrogen bond to corresponding deoxy-cytidilate subunits of the other strand.

FIGS. 2A-B illustrates the hydrogen bonding between the purine and pyrimidine bases of two anti-parallel DNA strands. FIG. 2A shows hydrogen bonding between adenine and thymine bases of corresponding adenosine and thymidine subunits, and FIG. 2B shows hydrogen bonding between guanine and cytosine bases of corresponding guanosine and cytosine subunits. Note that there are two hydrogen bonds 202 and 203 in the adenine/thymine base pair, and three hydrogen bonds 204-206 in the guanosine/cytosine base pair, as a result of which GC base pairs contribute greater thermodynamic stability to DNA duplexes than AT base pairs. AT and GC base pairs, illustrated in FIGS. 2A-B, are known as Watson-Crick (“WC”) base pairs.

Two DNA strands linked together by hydrogen bonds forms the familiar helix structure of a double-stranded DNA helix. FIG. 3 illustrates a short section of a DNA double helix 300 comprising a first strand 302 and a second, anti-parallel strand 304. The ribbon-like strands in FIG. 3 represent the deoxyribose and phosphate backbones of the two anti-parallel strands, with hydrogen-bonding purine and pyrimidine base pairs, such as base pair 306, interconnecting the two strands. Deoxy-guanylate subunits of one strand are generally paired with deoxy-cytidilate subunits from the other strand, and deoxy-thymidilate subunits in one strand are generally paired with deoxy-adenylate subunits from the other strand. However, non-WC base pairings may occur within double-stranded DNA.

Double-stranded DNA may be denatured, or converted into single stranded DNA, by changing the ionic strength of the solution containing the double-stranded DNA or by raising the temperature of the solution. Single-stranded DNA polymers may be renatured, or converted back into DNA duplexes, by reversing the denaturing conditions, for example by lowering the temperature of the solution containing complementary single-stranded DNA polymers. During renaturing or hybridization, complementary bases of anti-parallel DNA strands form WC base pairs in a cooperative fashion, leading to reannealing of the DNA duplex. Strictly A-T and G-C complementarity between anti-parallel polymers leads to the greatest thermodynamic stability, but partial complementarity including non-WC base pairing may also occur to produce relatively stable associations between partially-complementary polymers. In general, the longer the regions of consecutive WC base pairing between two nucleic acid polymers, the greater the stability of hybridization between the two polymers under renaturing conditions.

The ability to denature and renature double-stranded DNA has led to the development of many extremely powerful and discriminating assay technologies for identifying the presence of DNA and RNA polymers having particular base sequences or containing particular base subsequences within complex mixtures of different nucleic acid polymers, other biopolymers, and inorganic and organic chemical compounds. One such methodology is the array-based hybridization assay. FIGS. 4-7 illustrate the principle of the array-based hybridization assay. An array (402 in FIG. 4) comprises a substrate upon which a regular pattern of features is prepared by various manufacturing processes. The array 402 in FIG. 4, and in subsequent FIGS. 5-7, has a grid-like 2-dimensional pattern of square features, such as feature 404 shown in the upper left-hand corner of the array. Each feature of the array contains a large number of identical oligonucleotides covalently bound to the surface of the feature. These bound oligonucleotides are known as probes. In general, chemically distinct probes are bound to the different features of an array, so that each feature corresponds to a particular nucleotide sequence. In FIGS. 4-6, the principle of array-based hybridization assays is illustrated with respect to the single feature 404 to which a number of identical probes 405-409 are bound. In practice, each feature of the array contains a high density of such probes but, for the sake of clarity, only a subset of these are shown in FIGS. 4-6.

Once an array has been prepared, the array may be exposed to a sample solution of target DNA or RNA molecules (410-413 in FIG. 4) labeled with fluorophores, chemiluminescent compounds, or radioactive atoms 415-418. Labeled target DNA or RNA hybridizes through base pairing interactions to the complementary probe DNA, synthesized on the surface of the array. FIG. 5 shows a number of such target molecules 502-504 hybridized to complementary probes 505-507, which are in turn bound to the surface of the array 402. Targets, such as labeled DNA molecules 508 and 509, that do not contains nucleotide sequences complementary to any of the probes bound to array surface do not hybridize to generate stable duplexes and, as a result, tend to remain in solution. The sample solution is then rinsed from the surface of the array, washing away any unbound-labeled DNA molecules. In other embodiments, unlabeled target sample is allowed to hybridize with the array first. Typically, such a target sample has been modified with a chemical moiety that will react with a second chemical moiety in subsequent steps. Then, either before or after a wash step, a solution containing the second chemical moiety bound to a label is reacted with the target on the array. After washing, the array is ready for scanning. Biotin and avidin represent an example of a pair of chemical moieties that can be utilized for such steps.

Finally, as shown in FIG. 6, the bound labeled DNA molecules are detected via optical or radiometric scanning. Optical scanning involves exciting labels of bound labeled DNA molecules with electromagnetic radiation of appropriate frequency and detecting fluorescent emissions from the labels, or detecting light emitted from chemiluminescent labels. When radioisotope labels are employed, radiometric scanning can be used to detect the signal emitted from the hybridized features. Additional types of signals are also possible, including electrical signals generated by electrical properties of bound target molecules, magnetic properties of bound target molecules, and other such physical properties of bound target molecules that can produce a detectable signal. Optical, radiometric, or other types of scanning produce an analog or digital representation of the array as shown in FIG. 7, with features to which labeled target molecules are hybridized similar to 706 optically or digitally differentiated from those features to which no labeled DNA molecules are bound. In other words, the analog or digital representation of a scanned array displays positive signals for features to which labeled DNA molecules are hybridized and displays negative features to which no, or an undetectably small number of, labeled DNA molecules are bound. Features displaying positive signals in the analog or digital representation indicate the presence of DNA molecules with complementary nucleotide sequences in the original sample solution. Moreover, the signal intensity produced by a feature is generally related to the amount of labeled DNA bound to the feature, in turn related to the concentration, in the sample to which the array was exposed, of labeled DNA complementary to the oligonucleotide within the feature.

When a microarray is scanned, data may be collected as a two-dimensional digital image of the microarray, each pixel of which represents the intensity of phosphorescent, fluorescent, chemiluminescent, or radioactive emission from an area of the microarray corresponding to the pixel. A microarray data set may comprise a two-dimensional image or a list of numerical, alphanumerical pixel intensities, or any of many other computer-readable data sets. An initial series of steps employed in processing digital microarray images includes constructing a regular coordinate system for the digital image of the microarray by which the features within the digital image of the microarray can be indexed and located. For example, when the features are laid out in a periodic, rectilinear pattern, a rectilinear coordinate system is commonly constructed so that the positions of the centers of features lie as closely as possible to intersections between horizontal and vertical gridlines of the rectilinear coordinate system, alternatively, exactly half-way between a pair of adjacent horizontal and a pair of adjacent vertical grid lines. Then, regions of interest (“ROIs”) are computed, based on the initially estimated positions of the features in the coordinate grid, and centroids for the ROIs are computed in order to refine the positions of the features. Once the position of a feature is refined, feature pixels can be differentiated from background pixels within the ROI, and the signal corresponding to the feature can then be computed by integrating the intensity over the feature pixels.

Following exposure of a microarray to a sample solution, the entire feature-containing surface of the microarray may not be suitable for feature extraction for a variety of reasons. Portions of the array may be damaged by mishandling, portions of the array may be inadvertently contaminated or otherwise chemically modified during experimental procedures, there may be manufacturing defects present in portions of the microarray, and there may be other, similar problems that prevent portions of the microarray surface from being accurately scanned. Currently, when a user identifies damaged or defective portions of a microarray, the user needs to laboriously identify those features within the damaged, defective, or otherwise compromised subregions and manually edit a design file in order to eliminate the features within the compromised subregions from consideration by an automated feature extraction program. The manual, design-file-editing procedure is both time consuming and prone to error. For this reason, designers, manufactures, and users of microarray processing and feature extraction systems have recognized the need for a more user-friendly method for identifying features within compromised subregions of a microarray and removing those features from consideration by automated feature extraction programs.

### SUMMARY OF THE INVENTION

In one embodiment of the present invention, an automated microarray processing system displays, to a user, a visual rendering of the scanned image of a microarray, including putative feature locations, prior to undertaking automated feature extraction. The microarray processing system provides to the user an ability to draw one or more contour lines around those portions of the microarray considered by the user to be undamaged, non-defective, and otherwise not compromised and therefore suitable for feature extraction. The microarray processing system then constructs one or more rectangular regions of feature extractability based on the user-indicated subregions of feature extractability, and proceeds to extract data from the one or more rectangular regions of feature extractability.

### BRIEF DESCRIPTION OF THE DRAWINGS

FIG. 1 illustrates a short DNA polymer.

FIG. 2A shows hydrogen bonding between adenine and thymine bases of corresponding adenosine and thymidine subunits.

FIG. 2B shows hydrogen bonding between guanine and cytosine bases of corresponding guanosine and cytosine subunits.

FIG. 3 illustrates a short section of a DNA double helix.

FIGS. 4-7 illustrate the principle of array-based hybridization assays.

FIG. 8 shows a hypothetical computer display of a scanned image of a microarray.

FIG. 9 illustrates a visual display of a scanned image of a microarray containing two damaged or defective regions.

FIG. 10 illustrates editing of a design file.

FIG. 11 illustrates one method by which a user may identify a subregion of a microarray suitable for feature extraction.

FIG. 12 shows the subregion of FIG. 11, bounded by a contour line, at greater magnification, superimposed over a pixel grid.

FIGS. 13A-B illustrate nearest-neighbor analysis of pixels within the contour identified by a user as enclosing a subregion of a microarray suitable for feature extraction.

FIG. 14 illustrates the pixel intensities in pixels included in, and surrounding, a putative feature.

FIGS. 15-18 illustrate nearest-neighbor analysis for individual pixels within and near the putative feature, shown in FIG. 14, and in a defective or damaged region.

FIG. 19 illustrates a hypothetical bit mask prepared for the user-identified feature-extractable subregion of FIGS. 11, 12, and 13A-B.

FIG. 20 illustrates computation of the sums of the binary mask values along vertical columns with respect to the x and y coordinate axes shown in FIG. 19.

FIG. 21 illustrates summing of the binary-mask values within horizontal columns with respect to the x and y coordinate axes shown in FIG. 19.

FIG. 22 shows the bounding rectangle 2202 computed for the user-defined feature-extractable subregion 1902 within contour 1402.

FIG. 23 illustrates computation of a center of mass of the binary mask prepared from the user-defined feature-extractable subregion is computed.

FIG. 24 illustrates one approach to computing feature-extractable regions for multiple user-defined feature-extractable regions.

FIG. 25 is a control-flow diagram for the partial microarray technique described above with reference to FIGS. 11-25.

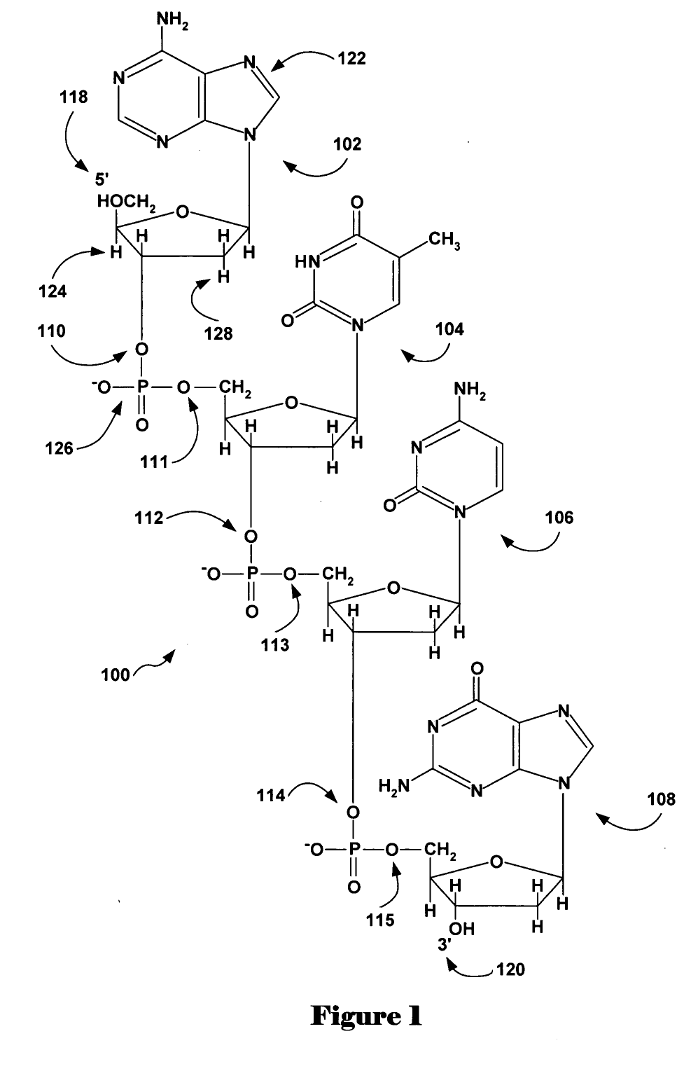

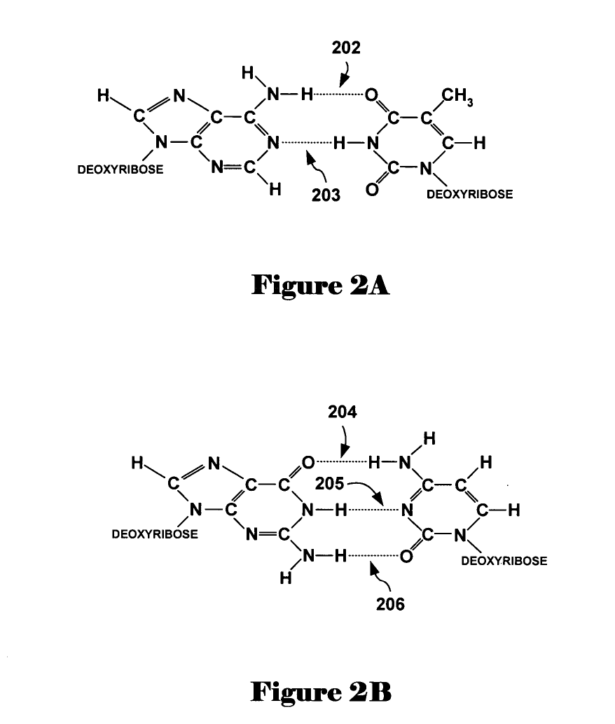

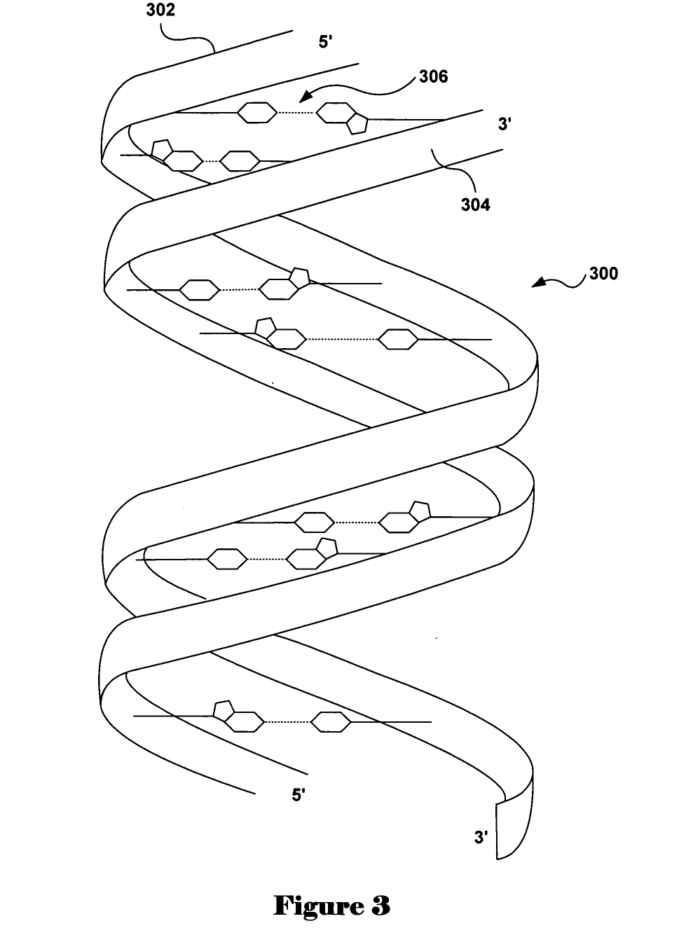

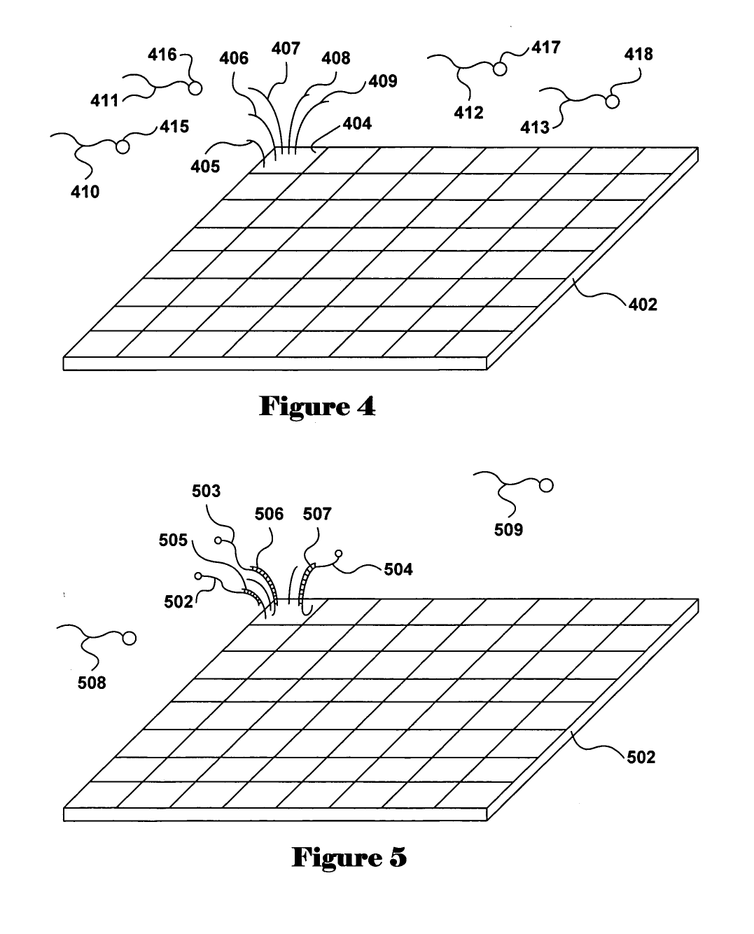

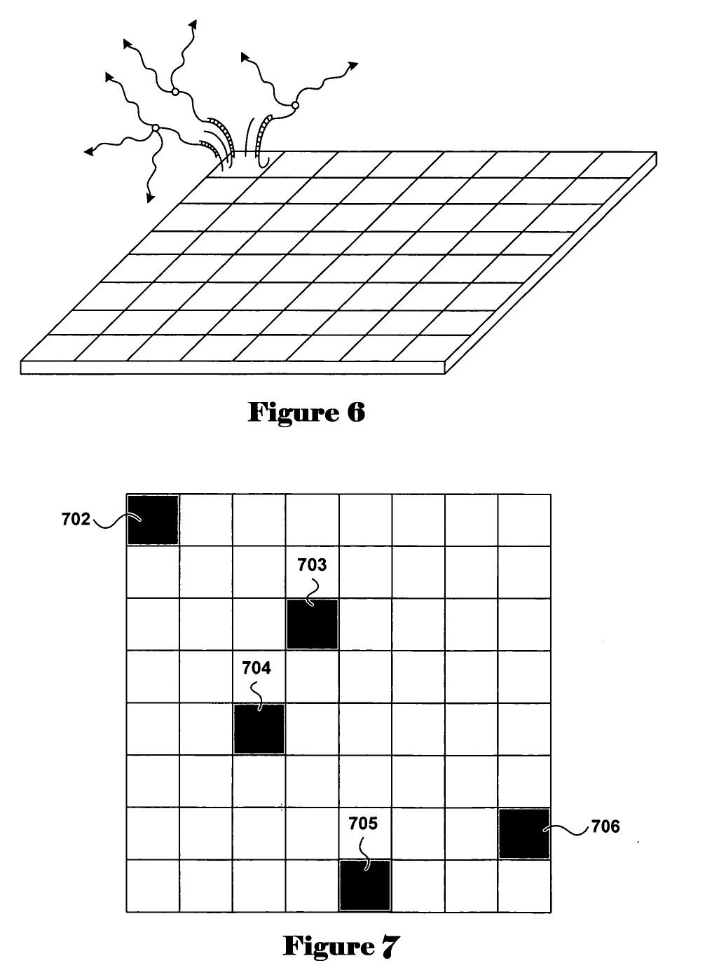

### DETAILED DESCRIPTION OF THE INVENTION

Various embodiments of the present invention allow a user to specify subregions of a microarray that the user feels are undamaged, non-defective, and otherwise non-compromised, and therefore suitable for automated feature extraction. Embodiments of the present invention are described, below, following a first subsection that provides additional information about microarrays.

### Additional Information About Microarrays

An array may include any one-, two- or three-dimensional arrangement of addressable regions, or features, each bearing a particular chemical moiety or moieties, such as biopolymers, associated with that region. Any given array substrate may carry one, two, or four or more arrays disposed on a front surface of the substrate. Depending upon the use, any or all of the arrays may be the same or different from one another and each may contain multiple spots or features. A typical array may contain more than ten, more than one hundred, more than one thousand, more ten thousand features, or even more than one hundred thousand features, in an area of less than 20 cm² or even less than 10 cm². For example, square features may have widths, or round feature may have diameters, in the range from a 10 μm to 1.0 cm. In other embodiments each feature may have a width or diameter in the range of 1.0 μm to 1.0 mm, usually 5.0 μm to 500 μm, and more usually 10 μm to 200 μm Features other than round or square may have area ranges equivalent to that of circular features with the foregoing diameter ranges. At least some, or all, of the features may be of different compositions (for example, when any repeats of each feature composition are excluded the remaining features may account for at least 5%, 10%, or 20% of the total number of features). Inter-feature areas are typically, but not necessarily, present. Inter-feature areas generally do not carry probe molecules. Such inter-feature areas typically are present where the arrays are formed by processes involving drop deposition of reagents, but may not be present when, for example, photolithographic array fabrication processes are used. When present, interfeature areas can be of various sizes and configurations.

Each array may cover an area of less than 100 cm², or even less than 50 cm², 10 cm² or 1 cm². In many embodiments, the substrate carrying the one or more arrays will be shaped generally as a rectangular solid having a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 and less than 1 mm. Other shapes are possible, as well. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, a substrate may transmit at least 20%, or 50% (or even at least 70%, 90%, or 95%), of the illuminating light incident on the front as may be measured across the entire integrated spectrum of such illuminating light or alternatively at 532 nm or 633 nm.

Arrays can be fabricated using drop deposition from pulsejets of either polynucleotide precursor units (such as monomers) in the case of in situ fabrication, or the previously obtained polynucleotide. Such methods are described in detail in, for example, U.S. Pat. No. 6,242,266, U.S. Pat. No. 6,232,072, U. S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. Other drop deposition methods can be used for fabrication, as previously described herein. Also, instead of drop deposition methods, photolithographic array fabrication methods may be used such as described in U.S. Pat. No. 5,599,695, U.S. Pat. No. 5,753,788, and U.S. Pat. No. 6,329,143. Interfeature areas need not be present particularly when the arrays are made by photolithographic methods as described in those patents.

A molecular array is typically exposed to a sample including labeled target molecules, or, as mentioned above, to a sample including unlabeled target molecules followed by exposure to labeled molecules that bind to unlabeled target molecules bound to the array, and the array is then read. Reading of the array may be accomplished by illuminating the array and reading the location and intensity of resulting fluorescence at multiple regions on each feature of the array. For example, a scanner may be used for this purpose, which is similar to the AGILENT MICROARRAY SCANNER manufactured by Agilent Technologies, Palo Alto, Calif. Other suitable apparatus and methods are described in U.S. patent applications: Ser. No. 10/087447 “Reading Dry Chemical Arrays Through The Substrate” by Corson et al., and Ser. No. 09/846125 “Reading Multi-Featured Arrays” by Dorsel et al. However, arrays may be read by any other method or apparatus than the foregoing, with other reading methods including other optical techniques, such as detecting chemiluminescent or electroluminescent labels, or electrical techniques, for where each feature is provided with an electrode to detect hybridization at that feature in a manner disclosed in U.S. Pat. No. 6,251,685, U.S. Pat. No. 6,221,583 and elsewhere.

A result obtained from reading an array, followed by application of a method of the present invention, may be used in that form or may be further processed to generate a result such as that obtained by forming conclusions based on the pattern read from the array, such as whether or not a particular target sequence may have been present in the sample, or whether or not a pattern indicates a particular condition of an organism from which the sample came. A result of the reading, whether further processed or not, may be forwarded, such as by communication, to a remote location if desired, and received there for further use, such as for further processing. When one item is indicated as being remote from another, this is referenced that the two items are at least in different buildings, and may be at least one mile, ten miles, or at least one hundred miles apart. Communicating information references transmitting the data representing that information as electrical signals over a suitable communication channel, for example, over a private or public network. Forwarding an item refers to any means of getting the item from one location to the next, whether by physically transporting that item or, in the case of data, physically transporting a medium carrying the data or communicating the data.

As pointed out above, array-based assays can involve other types of biopolymers, synthetic polymers, and other types of chemical entities. A biopolymer is a polymer of one or more types of repeating units. Biopolymers are typically found in biological systems and particularly include polysaccharides, peptides, and polynucleotides, as well as their analogs such as those compounds composed of, or containing, amino acid analogs or non-amino-acid groups, or nucleotide analogs or non-nucleotide groups. This includes polynucleotides in which the conventional backbone has been replaced with a non-naturally occurring or synthetic backbone, and nucleic acids, or synthetic or naturally occurring nucleic-acid analogs, in which one or more of the conventional bases has been replaced with a natural or synthetic group capable of participating in Watson-Crick-type hydrogen bonding interactions. Polynucleotides include single or multiple-stranded configurations, where one or more of the strands may or may not be completely aligned with another. For example, a biopolymer includes DNA, RNA, oligonucleotides, and PNA and other polynucleotides as described in U.S. Pat. No. 5,948,902 and references cited therein, regardless of the source. An oligonucleotide is a nucleotide multimer of about 10 to 100 nucleotides in length, while a polynucleotide includes a nucleotide multimer having any number of nucleotides.

As an example of a non-nucleic-acid-based molecular array, protein antibodies may be attached to features of the array that would bind to soluble labeled antigens in a sample solution. Many other types of chemical assays may be facilitated by array technologies. For example, polysaccharides, glycoproteins, synthetic copolymers, including block copolymers, biopolymer-like polymers with synthetic or derivitized monomers or monomer linkages, and many other types of chemical or biochemical entities may serve as probe and target molecules for array-based analysis. A fundamental principle upon which arrays are based is that of specific recognition, by probe molecules affixed to the array, of target molecules, whether by sequence-mediated binding affinities, binding affinities based on conformational or topological properties of probe and target molecules, or binding affinities based on spatial distribution of electrical charge on the surfaces of target and probe molecules.

Scanning of a molecular array by an optical scanning device or radiometric scanning device generally produces a scanned image comprising a rectilinear grid of pixels, with each pixel having a corresponding signal intensity. These signal intensities are processed by an array-data-processing program that analyzes data scanned from an array to produce experimental or diagnostic results which are stored in a computer-readable medium, transferred to an intercommunicating entity via electronic signals, printed in a human-readable format, or otherwise made available for further use. Molecular array experiments can indicate precise gene-expression responses of organisms to drugs, other chemical and biological substances, environmental factors, and other effects. Molecular array experiments can also be used to diagnose disease, for gene sequencing, and for analytical chemistry. Processing of molecular-array data can produce detailed chemical and biological analyses, disease diagnoses, and other information that can be stored in a computer-readable medium, transferred to an intercommunicating entity via electronic signals, printed in a human-readable format, or otherwise made available for further use.

### EMBODIMENTS OF THE PRESENT INVENTION

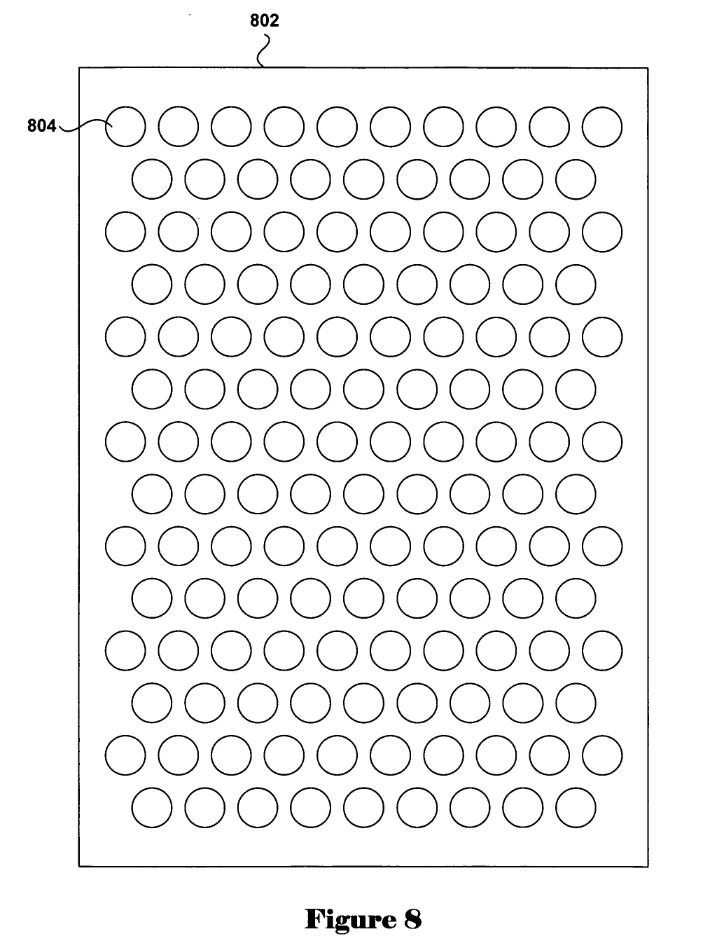

FIG. 8 shows a hypothetical computer display of a scanned image of a microarray. In the hypothetical scanned image of the microarray, shown in FIG. 8, a rectangular microarray 802 includes closely spaced features, such as feature 804, residing at putative feature positions obtained from a design file, or visible in a color or shading-based representation of the intensities of pixels within the scanned image of the microarray 802. Thus, the features indicated in the display shown in FIG. 8 may or may not correspond with actual features that would be extracted from the image by automated feature extraction procedures.

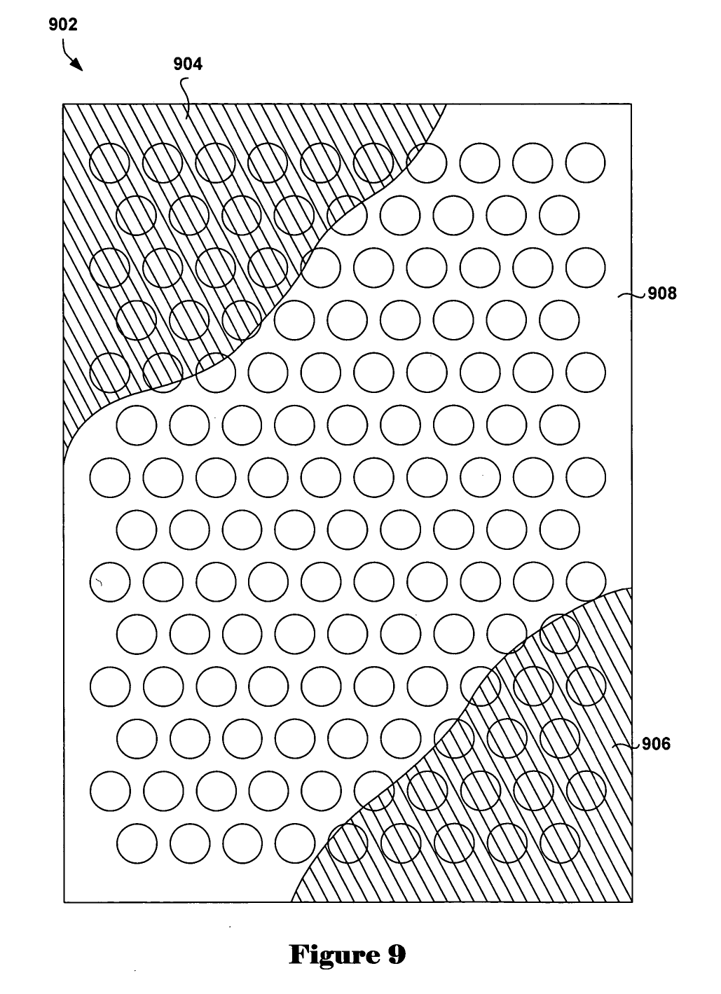

Following exposure of a microarray to a sample solution, the entire feature-containing surface of the microarray may not be suitable for feature extraction for a variety of reasons. Portions of the array may be damaged by mishandling, portions of the array may be inadvertently contaminated or otherwise chemically modified during experimental procedures, there may be manufacturing defects present in portions of the microarray, and there may be other, similar problems that prevent portions of the microarray surface from being accurately scanned. Often, the portions of a microarray that are not suitable for feature extraction may be visually identified by a user based on a visual display of the scanned image of the microarray. For example, FIG. 9 illustrates a visual display of a scanned image of a microarray when a computer screen, in which a user may readily identify two regions 904 and 906, shown in FIG. 9 with crosshatching, with intensities either lower, higher, or with different variation, than in the undamaged and undefective, viable portion of the microarray 908 from which accurate data can be extracted via an automated feature extraction program.

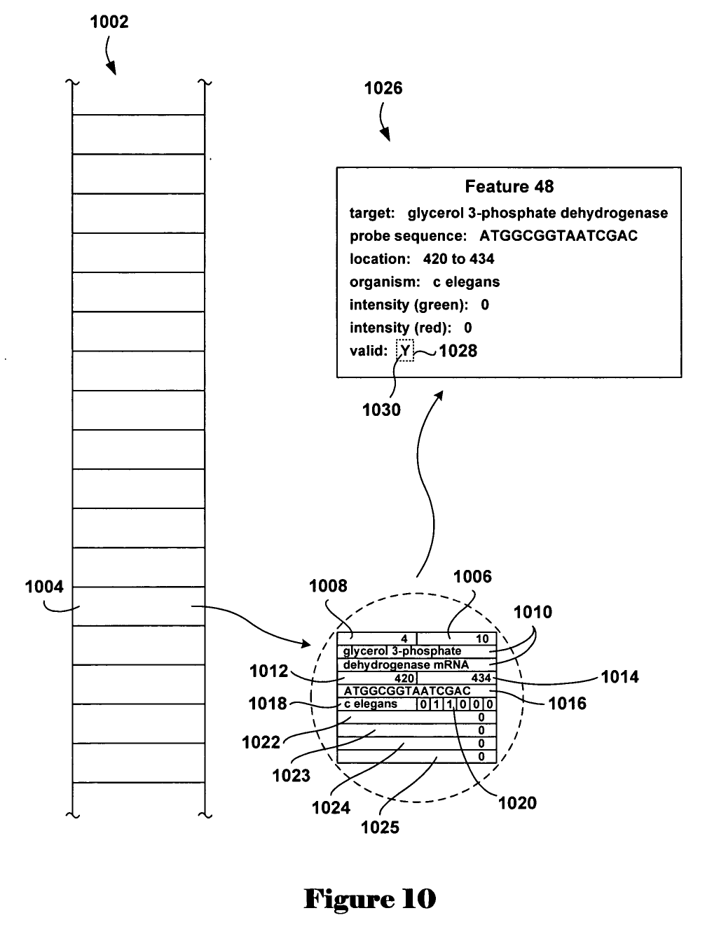

Currently, when a user identifies damaged or defective portions of a microarray, such as subregions 904 and 906 in the visual display of a scanned image of a microarray 902 in FIG. 9, the user needs to laboriously identify those features within the damaged, defective, or otherwise compromised subregions and manually edit a design file in order to eliminate the features within the compromised subregions from consideration by an automated feature extraction program. A design file is a computer file or computer database with records or entries for each feature in a microarray. FIG. 10 illustrates editing of a design file. In FIG. 10, the file or database is represented as a sequence of records, or entries 1002. A particular record or entry, such as record or entry 1004, may be accessed by feature number, feature index, a text rendering of the probe sequence or identity of the intended target for the feature, or by some other, similar means. The record or entry contains a number of fields, each field consisting of one or more computer-readable bits, bytes, words, or arrays of bits, bytes, or words. For example, a record or entry may include integer fields 1006 and 1008 specifying the indices of the feature within the microarray, a text field 1010 containing the name of the target of the feature, integer fields that identify the subsequence of the target to which the probe molecule in the feature is complementary 1012 and 1014, a text field including the probe sequence 1016, a text field identifying the organism from which the target molecule is obtained 1018, a number of bit fields, such as bit field 1020 that indicates whether or not the feature is valid, or useable, and additional fields 1022-1025 which are used to store signal data for the feature following automated feature extraction. The computer-readable record or entry 1004 may be rendered for visual display 1026 and displayed to a user by a design-file editor. In order to remove features contained within damaged or defective subregions of a microarray, a user currently needs to identify the features within those compromised subregions, access the records or entries corresponding to the features through a design-file editor, and edit the contents of the record or feature to indicate that the feature should not be considered by an automated feature extraction program. In the example of FIG. 10, the user would move a cursor 1028 to highlight an alphanumeric 1030 rendering of a bit field in order to designate the feature as not valid.

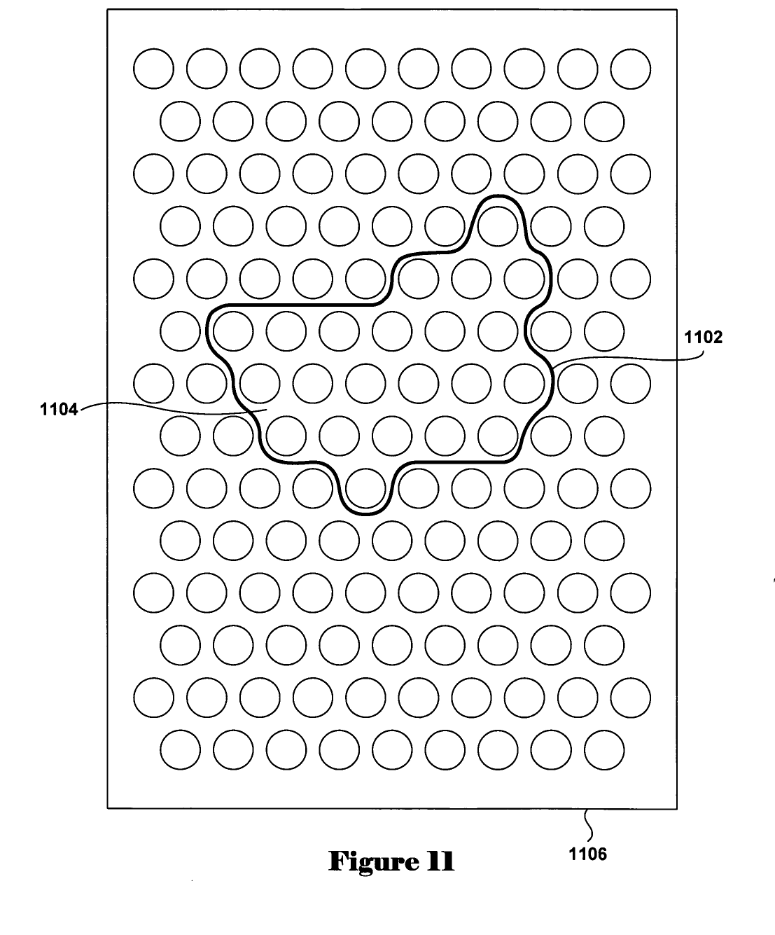

Various embodiments of the present invention allow a user to identify one or more subregions of a microarray suitable for feature extraction. FIG. 11 illustrates one method by which a user may identify a subregion of a microarray suitable for feature extraction. In FIG. 11, a user has drawn a contour line 1102 about a subregion 1104 of a microarray visually displayed 1106 on a computer screen. In one embodiment, the user employs a touchscreen to manually draw the one or more contour lines directly on the visually displayed image of a microarray. In alternative embodiments, navigational keys on a computer keyboard are used to initiate, steer, and terminate entry of a contour line via cursor movement. Many other alternative means for providing a user the ability to input a contour line may be employed in additional, alternative embodiments. It should be noted that thousands or tens of thousands of putative feature positions may be present within the scanned image of a microarray. Therefore, visual display of the scanned image of a microarray normally involves zooming operations, allowing a user to visualize the entire scanned image of the microarray, to navigate to particular portions of the microarray, and to change the scale of presentation in order to view putative feature positions at magnifications levels at which individual pixels are evident. FIGS. 8, 9, and 11 employ a very small number of features for clarity of illustration. Note also that the drawing of a contour line by a user may involve a number of navigational and zooming operations to allow the user to precisely place the contour line to surround one or more regions that appear to be suitable for feature extraction.

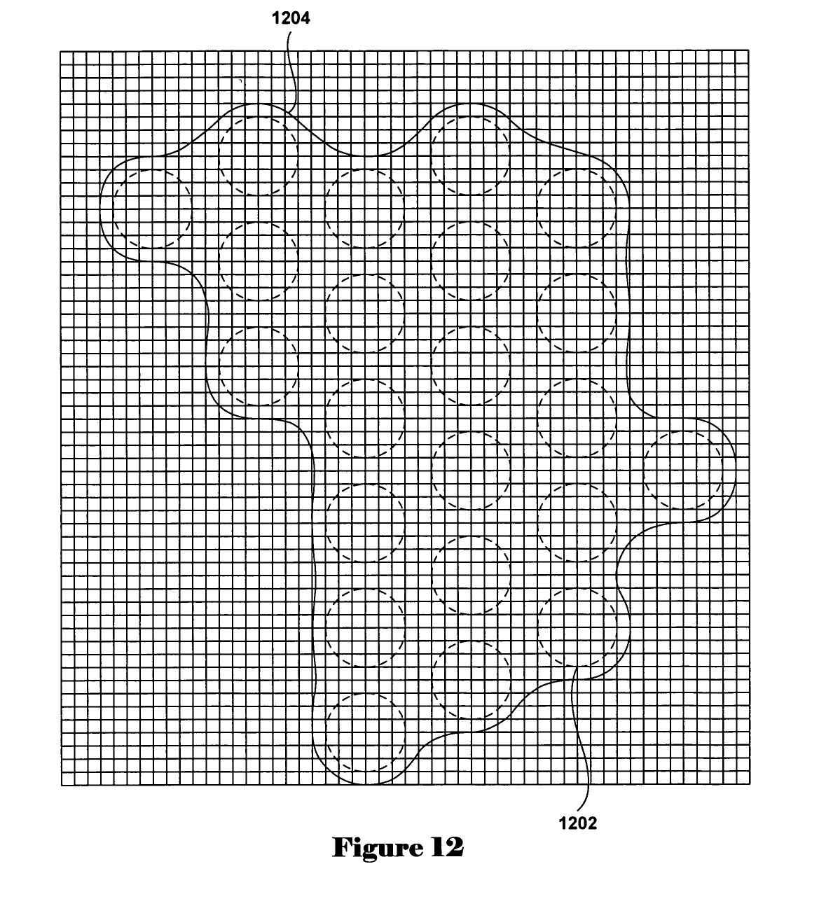

FIG. 12 shows the subregion bounded by a contour line, shown in FIG. 11, at greater magnification, superimposed over a pixel grid. Again, in a normal microarray, each putative feature location may include a much larger number of pixels than the putative features, indicated in FIG. 12 by circular dashed lines, such as circular dashed line 1202. A relatively large pixel grid, and a correspondingly small number of pixels per putative feature, are used in FIG. 12 and subsequent figures for clarity of illustration. The subregion suitable for feature extraction 1204 has been rotated by 90 degrees in a counter-clockwise direction from the display in FIG. 11.

Embodiments of the present invention employ pixel-based analysis techniques in order to transform an irregularly shaped region identified by a user as suitable for feature extraction, such as region 1204 in FIG. 12, into an easily described, regular region, such as a rectangular region. Irregular regions are not easily described mathematically or algorithmically, and often have low symmetry, two quite related aspects. Quite often, an irregular region needs to be described by a curved perimeter, generally by a large set of points at reasonable intervals along the perimeter, the interval length needed, or needed resolution, depending on the maximum curvature of the perimeter. Regular regions, by contrast, have relatively high symmetry, and can be easily described mathematically and/or algorithmically. For example, it is very difficult to determine an analytical function or a simple algorithm to describe or construct a misshapen blob. By contrast, a square can be simply described by the coordinates of two, particular vertices.

In certain embodiments of the present invention, a bit mask, with each bit representing a single pixel within the scanned image of the microarray, is prepared for the identified subregion or subregions suitable for feature extraction. The bit map is prepared by successive analysis of each pixel within the scanned image of the microarray. As discussed above, each pixel in the scanned image of a microarray represents a square or rectangular subregion of the microarray and is associated with an intensity value, for a one-channel microarray, or a number of intensity values for a multi-channel microarray. In the following, analysis of a subregion is described with reference to a single intensity value, or single channel, for each pixel. In alternative embodiments, separate analyses may be undertaken for each channel, or set of intensity values, and the intersection of the resulting rectangular regions employed for feature extraction. In other alternative embodiments, the intensity signals may be combined to produce a combined intensity signal on which the analysis, described below, is carried out. In additional, alternative embodiments, separately determined rectangular subregions suitable for feature extractability in each channel may be used for feature extraction of the corresponding intensity sets, resulting in some number of features extracted in only one, or a subset of, multiple channels or intensity sets.

In one technique for intensity-based analysis, the intensities of a number of neighboring pixels within a square neighborhood of a pixel under consideration are considered in order to determine whether or not the pixel under consideration should be set to the binary value “1” in a bit mask, or set to the binary value “0.” Either of two binary conventions can be used. In the current discussion, a binary value “1” indicates that the pixel appears, based on the intensities of its neighbors, to be included in a subregion of the microarray suitable for feature extractability. The neighborhood for a pixel may include the eight nearest neighbors within a square region centered about the considered pixel, may consist of the 24 nearest in a square region centered about the considered pixel, or may consist of some other number of nearest neighbor pixels in a more complex area that includes the considered pixel.

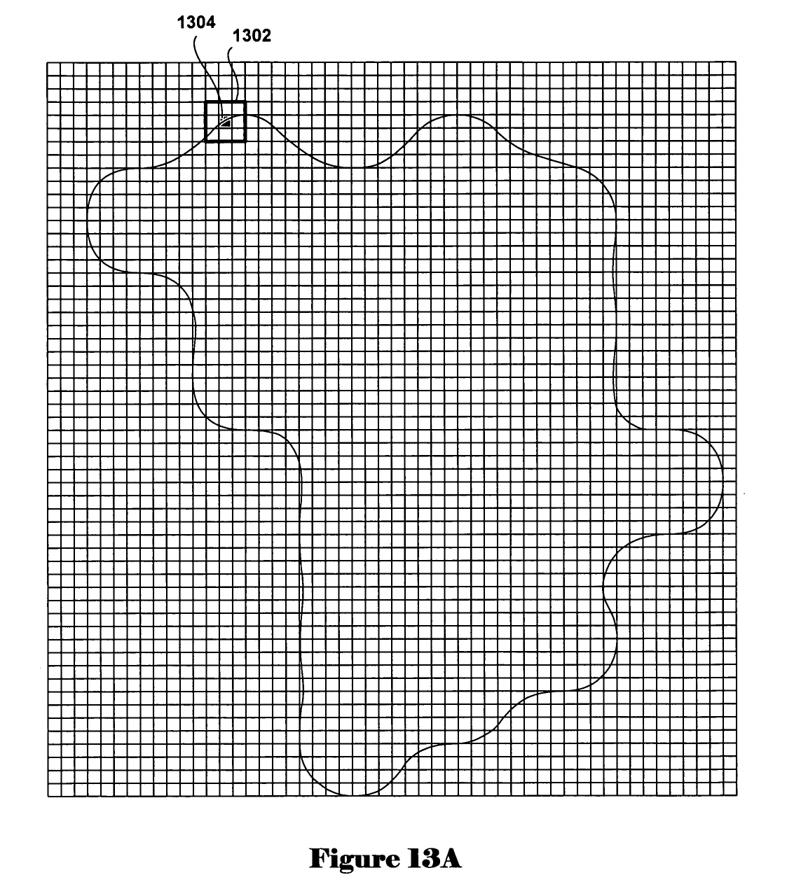

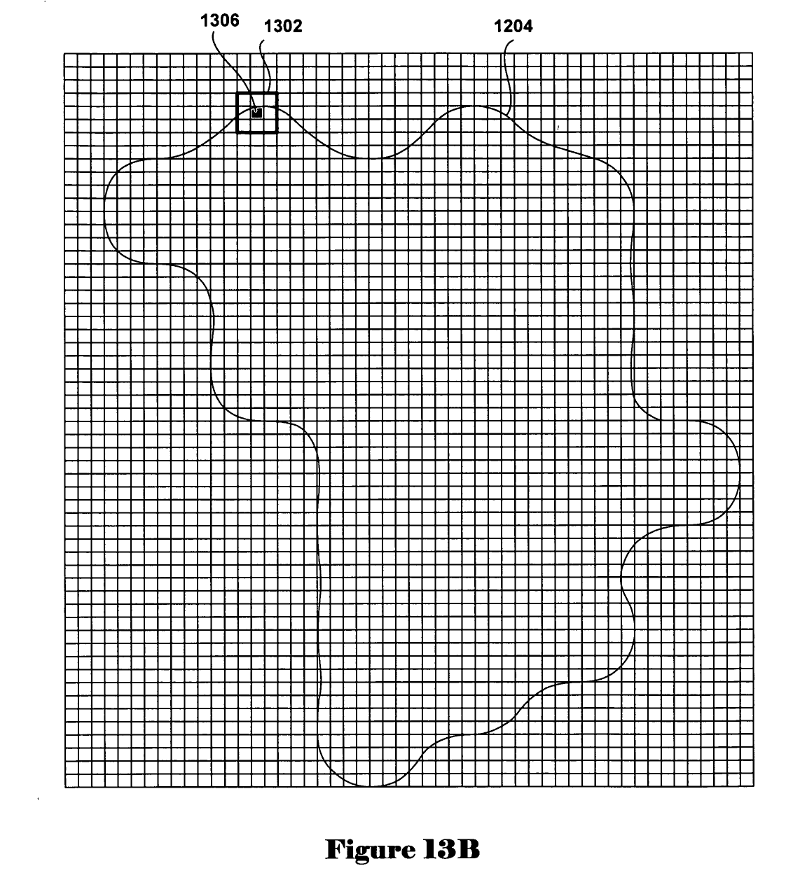

FIGS. 13A-B illustrate nearest-neighbor analysis of pixels within the contour identified by a user as enclosing a subregion of a microarray suitable for feature extraction. The pixels within the contour are each considered in a normal, left-to-right, top-down, raster-like scan of the region bounded by the contour line. As shown in FIG. 13A, the eight nearest neighbors within a square region 1302 centered about the first pixel 1304 encountered in the raster-like scan of the region within the contour are considered in order to determine whether or not the value for the first pixel 1304 in the binary mask should be set to “1” or “0.” Then, in a next step of the raster-like scab, as shown in FIG. 13B, the nearest-neighbor square 1302 is shifted one pixel to the right in order to consider a next pixel 1306. The nearest-neighbor analysis proceeds with each subsequent pixel within the subregion enclosed by the contour line 1204.

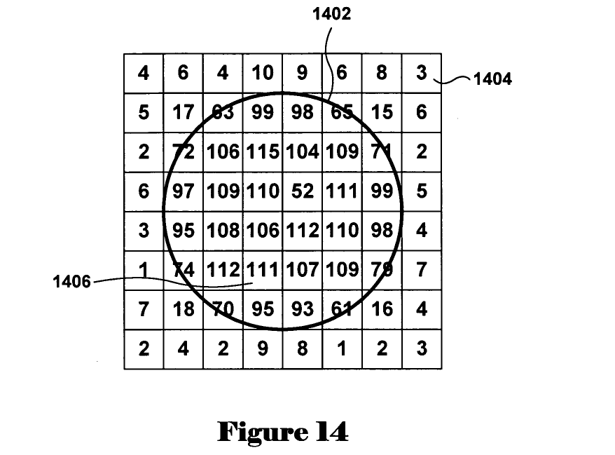

FIG. 14 illustrates the pixel intensities in pixels included in, and surrounding, a putative feature. In FIG. 14, the putative feature location is indicated by circle 1402. The number in each square of the grid, such as the number “3” in square 1404, represents the intensity value for the pixel represented by the square within the grid. Note that the intensities of pixels within the putative feature area, such as the intensity “111” within pixel 1406, are generally higher than the intensities of the pixels outside the putative feature area. In other words, the putative feature area 1402 in fact corresponds to an area of a feature in the scanned image of a microarray.

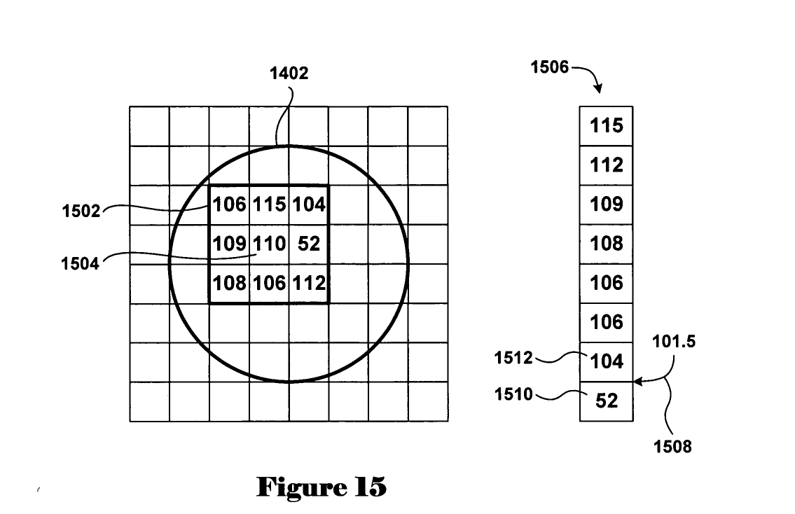

FIGS. 15-18 illustrate nearest-neighbor analysis for individual pixels within and near the putative feature, shown in FIG. 14, and in a defective or damaged region. In FIG. 15, the neighborhood 1502 of a pixel 1504 within the putative feature 1402 is considered. The intensities of the eight neighbors of the central pixel 1504 within the neighborhood 1502 are sorted in the array 1506 by intensity value. The average pixel intensity value for the eight nearest neighbors of pixel 1504 is computed as “101.5.” Then, the array of pixel-intensity values 1506 is searched to find a position within the array representing the computed average value “101.5.” As shown in FIG. 15, that position 1508 falls between the lowest pixel intensity value 52 (1510) and the next lowest pixel intensity value “104” (1512). In a number of embodiments of the present invention, the decision whether to include a pixel, such as pixel 1504, in the binary mask computed from the pixel intensity data or, in other words, to set the bit corresponding to the pixel within the binary mask to binary value “1,” is determined by the position of the computed average value within the sorted array. In the currently described embodiment, if at least half of the pixel-intensity values in the sorted array are greater than the computed average value, then the binary value for the considered pixel is set to binary value “1.” Otherwise, the binary value for the considered pixel is set to binary value “0.” In the case of FIG. 15, all but one of the nearest neighbor pixel intensity values are greater than the computed average value, and so the binary value “1” would be set in the bit of the bit mask corresponding to pixel 1504.

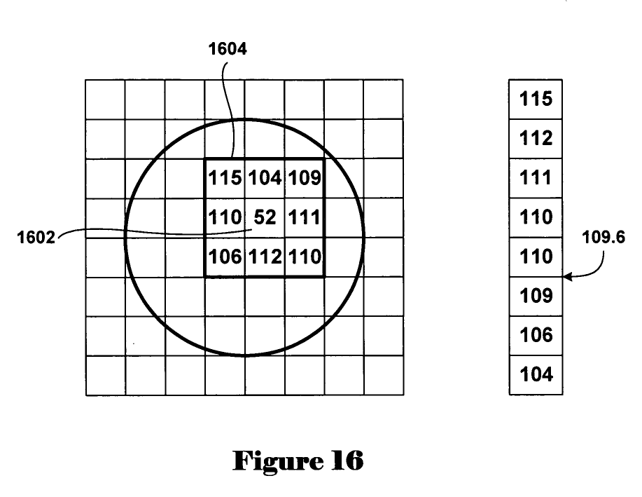

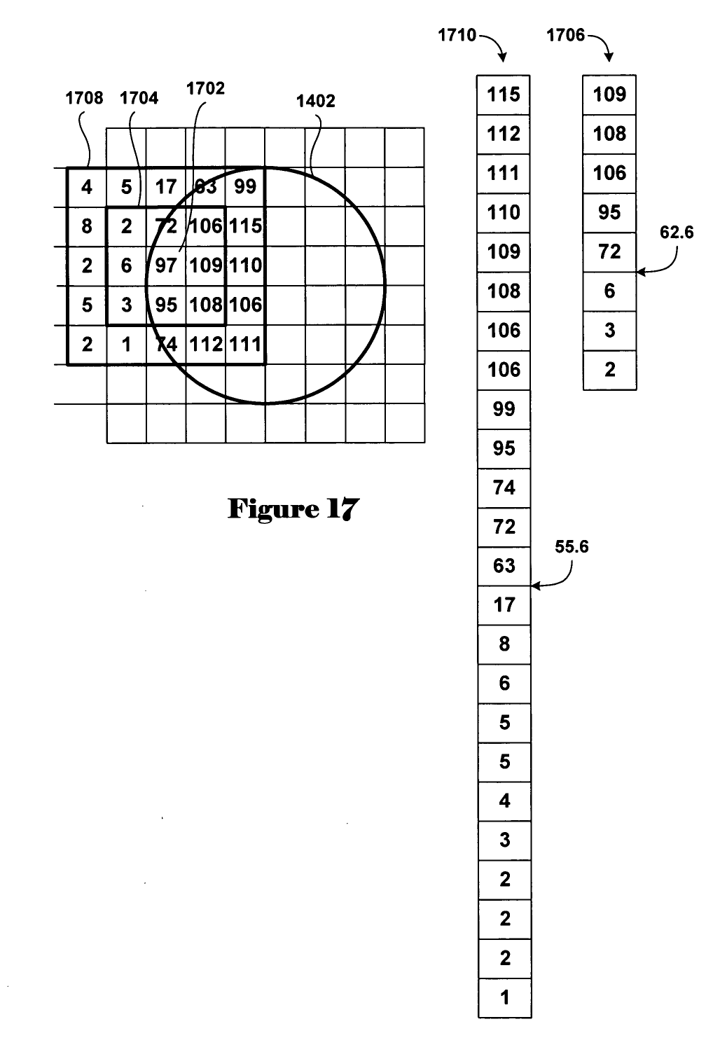

FIG. 16 illustrates nearest neighbor analysis for central pixel 1602 within neighborhood 1604. Again, more than half of the nearest neighbor pixel intensities are greater than the computed average intensity “109.6.” FIG. 17 illustrates the nearest neighbor analysis for a pixel 1702 near the edge of the putative feature 1402. An eight-neighbor nearest neighbor analysis based on neighborhood 1704 is shown by the sorted array 1706 of pixel intensity values from the eight-neighbor neighborhood 1704. More than half of the intensities associated with the eight nearest neighbor pixels have intensity values greater than the computed average pixel intensity value for the eight nearest neighbors “62.6.” Alternatively, a nearest neighbor analysis may be conducted over the neighborhood 1708 that includes 24 nearest neighbors of the central pixel 1702. The pixel intensities of the 24 nearest neighbors are sorted into array 1710. Again, more than half of the pixel intensity values are greater than the computed average pixel intensity value “55.6” for the 24 nearest neighbors. Thus, central pixel 1702 would have the corresponding binary bit-mask value “1.”

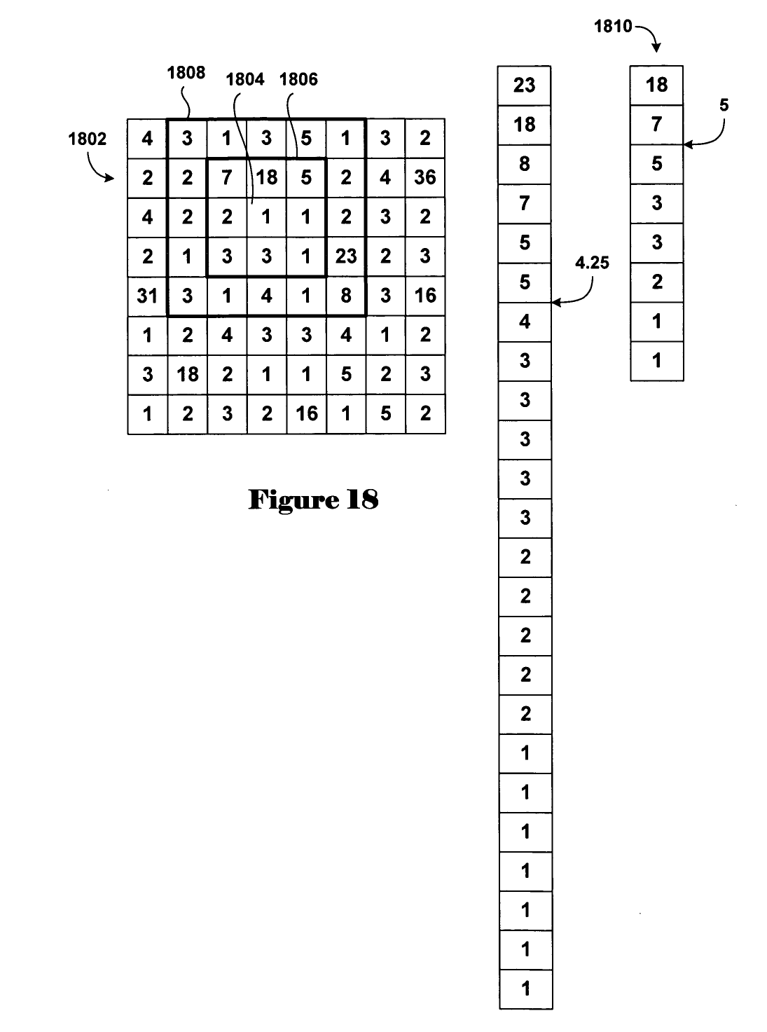

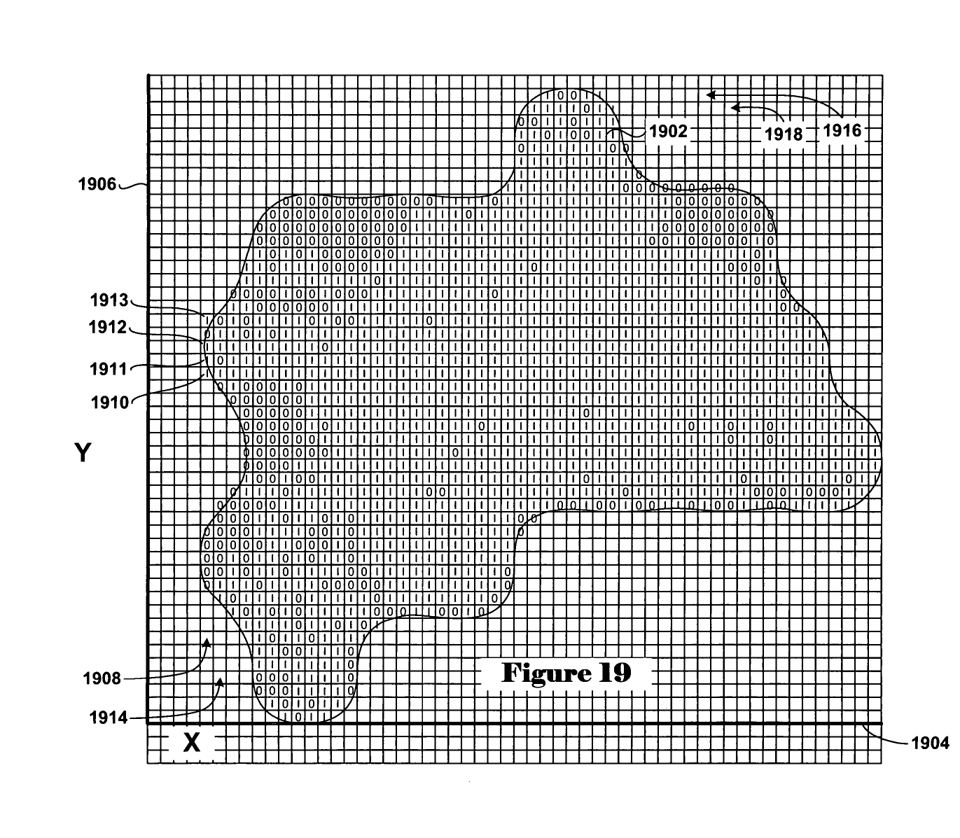

FIG. 18 shows a small, square area 1082 of pixels from a subregion of the microarray that is damaged, defective, or otherwise unsuitable for feature extraction. As can be seen in FIG. 18, the pixel intensity values within the region have a rather skewed distribution, with many low-intensity pixels and a few, randomly distributed, rather higher intensity pixels. Nearest neighbor analysis for pixel 1804 based on either the eight-neighbor neighborhood 1806 or the 24-neighbor neighborhood 1808 results in a determination that the central pixel 1804 should have a corresponding binary value “0” in the binary mask. Only two of six pixel intensity values for the eight nearest neighbors of neighborhood 1806 have values greater than the computed average pixel intensity for the eight nearest neighbors, shown sorted in array 1810, and only six of the 24 nearest neighbors of neighborhood 1808 have pixel intensity values greater than the computed average pixel intensity value “4.25.”

In the above-described embodiment, the determination of whether a pixel belongs to a feature-extractable subregion or not is related to whether or not the computed average pixel intensity value for the nearest neighbors of the pixel is equal to, or less than, the median pixel intensity value. This method, or metric, is tailored to identifying pixels within regions, such as the region 1802 in FIG. 18, in which a few relatively high intensity pixels are scattered amongst a large number of relatively low-intensity pixels. This type of pixel intensity distribution is symptomatic of damaged or defective microarray subregions for a class of microarrays for which the above-described embodiment has been developed. Other types of nearest neighbor analyses may be employed in alternative embodiments. For example, the decision as to whether include or exclude a particular pixel from the binary mask may be based on the variance of pixel intensities within a neighborhood including the pixel, on a comparison of the pixel's intensity with the intensities of its nearest neighbors, or on any number of other types of tests or metrics that appropriately discriminate, based on neighboring pixels, pixels belonging to feature-extractable subregions from pixels belonging to defective or damaged subregions. In additional, alternative embodiments, more global approaches to the analysis may be used, including employing larger neighborhoods, computing bit-mask values for groups of pixels, and other techniques.

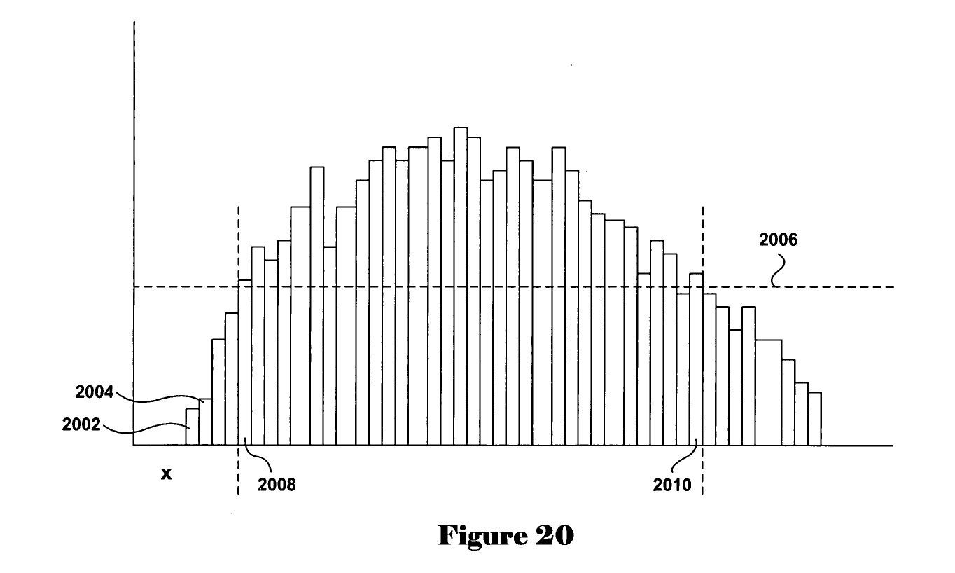

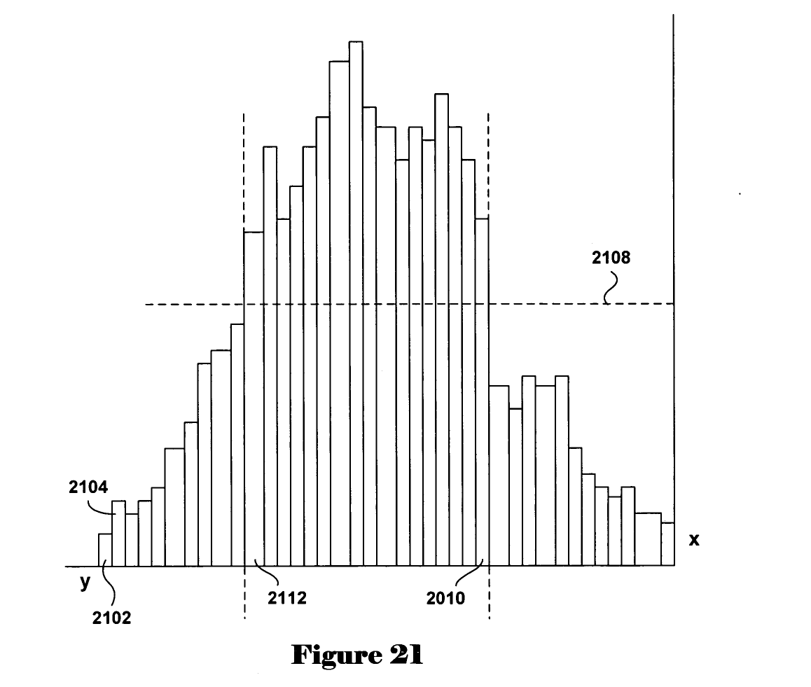

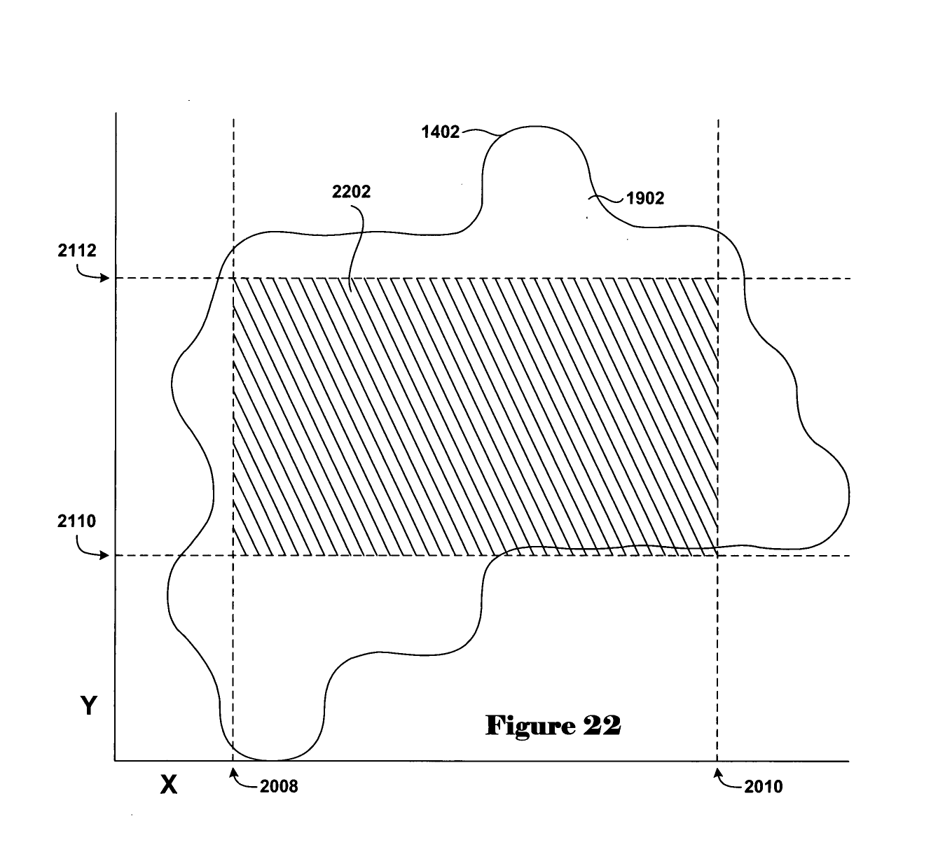

Whether by eight-neighbor nearest neighbor analysis, 24-neighbor nearest neighbor analysis, or other pixel-intensity analyses, a bit mask for the feature-extractable subregion or subregions identified by a user are prepared. FIG. 19 illustrates a hypothetical bit mask prepared for the user-identified feature-extractable subregion of FIGS. 11, 12, and 13A-B. As shown in FIG. 19, each pixel within a user-identified feature-extractable subregion 1902 has been assigned either the binary value “1” or the binary value “0.” A horizontal axis x 1904 and a vertical axis y 1906, both incremented in pixels, are assigned within the pixel grid to allow for local indexing of each pixel within the binary mask. FIG. 20 illustrates computation of the sums of the binary mask values along vertical columns with respect to the x and y coordinate axes shown in FIG. 19. For example, the column of height 4 2002 appearing in FIG. 20 corresponds to vertical pixel column 1908 in FIG. 19 which includes four pixels having the binary value “1” 1910-1913. Similarly, column 2004 in FIG. 20 corresponds to the sum of the values in vertical column 1914 in FIG. 19. FIG. 21 illustrates summing of the binary-mask values within horizontal columns with respect to the x and y coordinate axes shown in FIG. 19. For example, column 2102 in FIG. 21 of height 3 corresponds to the sum of the binary-mask values in horizontal row 1916 in FIG. 19. Similarly, column 2104 in FIG. 21, of height 6, corresponds to the sum of the binary-mask values in horizontal row 1918. The width and height of a bounding rectangle are computed from the plots of column and row sums shown in FIGS. 20 and 21, respectively. In both cases, one half of a height of the largest column or row value is calculated, and a horizontal line of that height, 2006 and 2108, respectively, is plotted. The left-hand position of the bounding rectangle is obtained as the first column, in FIG. 20, having a value greater than the one-half maximum column height value, column 2008. The right-hand position for the bounding rectangle is computed as the final column with height greater than the one-half maximum height value, column 2010. Similarly, as shown in FIG. 21, the lower-most boundary for the bounding rectangle is computed as the position of the first column 2110 with height greater than the computed one-half maximum height 2108, and the upper position of the bounding rectangle is computed as the final column 2112 with height greater than the computed one-half maximum height 2108.

FIG. 22 shows the bounding rectangle 2202 computed for the user-defined feature-extractable subregion 1902 within contour 1402. The bounding rectangle is shown in FIG. 22 as a crosshatched rectangle 2202. The x-axis positions of the left and right vertical sides of the bounding rectangle 2202 correspond to the computed x-coordinate positions 2008 and 2010 shown in FIG. 20, and the y-axis positions of the lower and upper edges of the bounding rectangle correspond to the computed y-coordinate positions 2110 and 2112 in FIG. 21. The bounding rectangle 2202 corresponds to a rectangular subregion with the greatest density of binary values “1” within the binary mask. A rectangular subregion is computed because a rectangular subregion is easily described by two x-coordinate and two y-coordinate values 2008, 2010, 2110, and 2112 in FIG. 22.

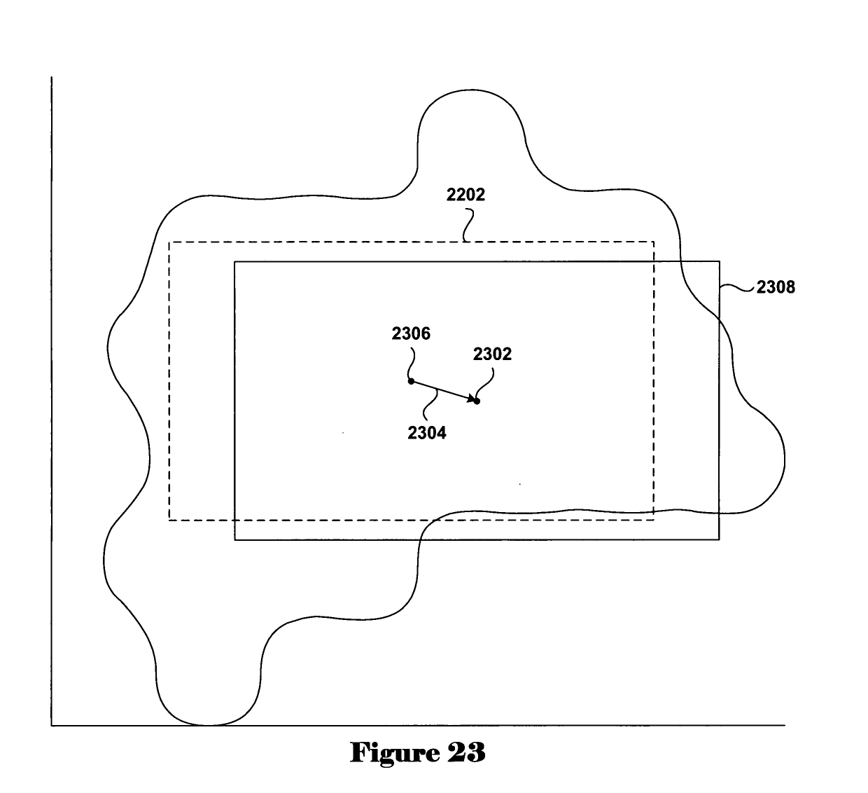

Next, as shown in FIG. 23, the center of mass of the binary mask prepared from the user-defined feature-extractable subregion is computed. The coordinates (x_{c}, y_{c}) of the center of mass may be computed as:

$$\begin{matrix}{x}_{c} = \frac{{\sum }_{i = 1}^{n} {x}_{i} {M}_{i}}{n} \\ {y}_{c} = \frac{{\sum }_{i = 1}^{n} {y}_{i} {M}_{i}}{n}\end{matrix}$$

where xᵢ and yᵢ are the x and y coordinates for the binary mask value corresponding to pixel i, n is the number of pixels within the user-defined subregion, and Mᵢ is the value in the binary mask corresponding to pixel i. In FIG. 23, the center of mass of the binary mask prepared from the user-defined feature-extractable subregion coincides with point 2302. The originally computed bounding rectangle 2202 is then moved so that the center of the bounding rectangle coincides with the computed center of mass 2302. As shown in FIG. 23, the adjustment of the position of the bounding box coincides with a vector displacement 2304 of the geometric center of the originally computed bounding rectangle 2306 to the computed center of mass 2302 of the binary mask prepared from the user-defined subregion. The final bounding rectangle 2308 is shown in FIG. 23 with solid edges. Feature signals, or, in other words, integrated, background-subtracted pixel intensities over regions of a scanned image of a microarray corresponding to features, are then computed, by an automated feature-extraction program, from the bounding rectangle 2308.

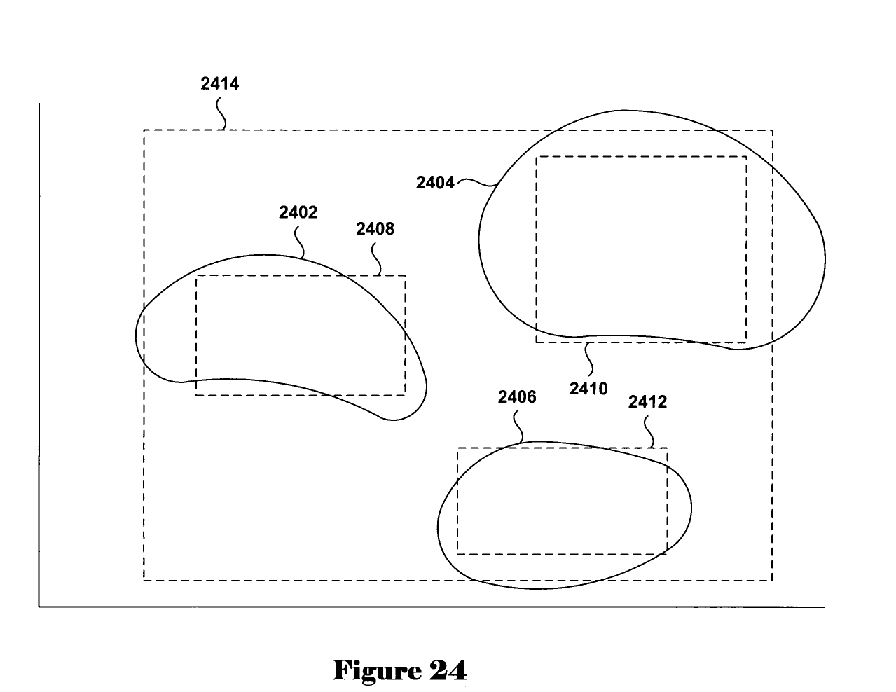

FIG. 24 illustrates one approach to computing feature-extractable regions for multiple user-defined feature-extractable regions. In FIG. 24, the user has drawn three separate contours 2402, 2404, and 2406 enclosing subregions that the user considers to be feature extractable. As shown in FIG. 24, the above-described technique may be employed for each separate feature-extractable subregion in order to determine a bounding rectangle 2408, 2410, and 2412 for each separate feature-extractable subregion. An automated feature-extraction program can then extract signals from the three bounding rectangles 2408, 2410, and 2412. In alternative embodiments, a single bounding rectangle 2414 may be computed based on a single bit mask prepared based on the three feature-extractable subregions defined by a user. In other words, rather than treating each separate user-defined feature-extractable subregion separately, the three subregions may be employed, together, to prepare a single bit mask, from which a single bounding rectangle 2414 may be prepared. The latter approach provides greater simplicity at the expense of potentially including a rather large amount of damaged or defective microarray surface area.

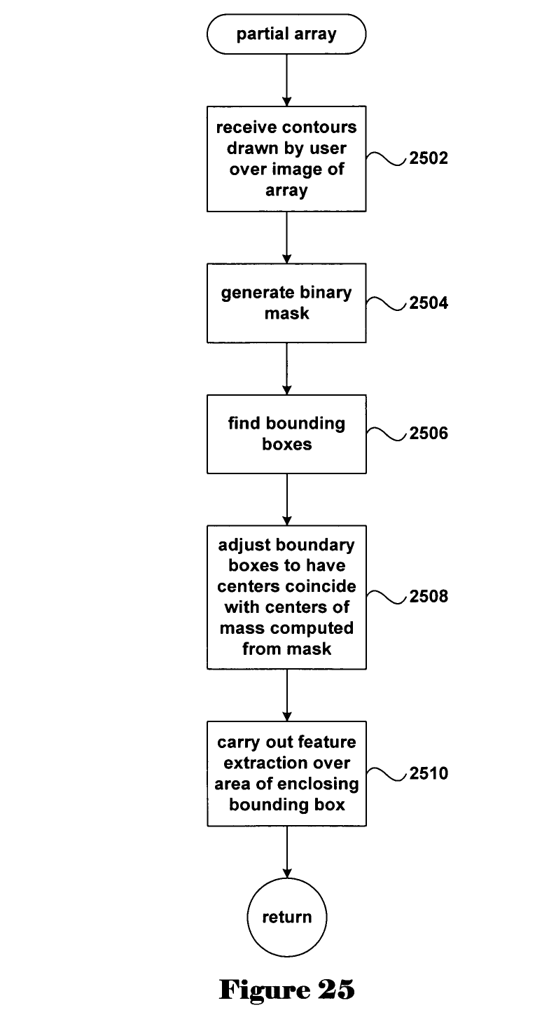

FIG. 25 is a control-flow diagram for the partial microarray technique described above with reference to FIGS. 11-25. In the first step, a partial-array routine of a microarray data processing program receives one or more contours drawn by a user to indicate feature-extractable subregions within a displayed image of a microarray, in step 2502. Contour input is described above, with reference to FIG. 11. Next, in step 2504, the partial-array routine generates, in one embodiment, a binary mask for each separate feature-extractable region identified by the user or, in another embodiment, a single binary mask encompassing all feature-extractable subregions identified by the user. Binary mask generation is described above with reference to FIGS. 12-19. Then, in step 2506, the partial-array routine determines either a separate bounding box for each user-identified feature-extractable region, or a single bounding box encompassing all user-identified feature-extractable subregions by a construction process discussed above with reference to FIGS. 20-22. Next, in step 2508, the partial-array routine adjusts the position or positions of the one or more bounding boxes so that the geometric center of each bounding box coincides with the center of mass or centers of mass computed for the one or more binary masks. In one embodiment, each bounding box computed for each separate user-defined feature-extractable subregion is adjusted to have its geometric center coincide with a center of mass computed from a separate binary mask for the user-defined feature-extractable subregion. In an alternative embodiment, a single bounding rectangle is moved with respect to a pixel grid so that the geometric center of the single bounding box coincides with a center of mass computed for a single binary mask encompassing all of the user-defined subregions. Finally, in step 2510, the microarray data processing system invokes an automated feature extraction routine to carry out the signal extraction from the feature-extractable subregions defined by one or more bounding boxes created in positions in steps 2506 and 2508.

Although the present invention has been described in terms of a particular embodiment, it is not intended that the invention be limited to this embodiment. Modifications within the spirit of the invention will be apparent to those skilled in the art. For example, as discussed above, any number of nearest neighbor analysis techniques may be employed for creation of a binary mask from one or more user-defined feature-extractable subregions within the image of the microarray. Although a one-half maximum column or row value is employed, in the above-described embodiment, to compute the positions of the edges of the bounding box, alternative approaches may be employed, including inscribing the user-definer feature-extractable subregion or subregions within a rectangle. As discussed above, when a user defines more than one feature-extractable subregion, the individual subregions may be treated separately, or treated together by forming a single binary mask. The x and y axes within the pixel grid may be rather arbitrarily assigned, or may be assigned in order to partially inscribe a user-defined feature-extractable region within the positive quadrant. The above described embodiments employed bounding boxes for specifying regions of feature extractability, but bounding disks and other easily constructed shapes may be alternatively employed. A suitable bounding shape is one that can be constructed from one or a few parameters, and for which pixel membership can be computationally efficiently determined.

The foregoing description, for purposes of explanation, used specific nomenclature to provide a thorough understanding of the invention. However, it will be apparent to one skilled in the art that the specific details are not required in order to practice the invention. The foregoing descriptions of specific embodiments of the present invention are presented for purpose of illustration and description. They are not intended to be exhaustive or to limit the invention to the precise forms disclosed. Obviously many modifications and variations are possible in view of the above teachings. The embodiments are shown and described in order to best explain the principles of the invention and its practical applications, to thereby enable others skilled in the art to best utilize the invention and various embodiments with various modifications as are suited to the particular use contemplated. It is intended that the scope of the invention be defined by the following claims and their equivalents:

1. A method for processing microarray data, the method comprising:
rendering the microarray data for visual display;
displaying the microarray data rendered for visual display;
receiving as input a boundary of a region of feature extractability within the microarray;
constructing a regularly shaped region of feature extractability from the received boundary of the region of feature extractability within the microarray; and
extracting feature signals from the regularly shaped region of feature extractability.

2. The method of claim 1 wherein rendering the microarray data for visual display further includes preparing a pixel-based, scanned image of the microarray with indications of putative feature positions.

3. The method of claim 2 wherein displaying the microarray data rendered for visual display further includes displaying, on a computer display device, the pixel-based, scanned image of the microarray with indications of putative feature positions.

4. The method of claim 1 wherein receiving a boundary of a region of feature extractability within the microarray further includes receiving a contour line enclosing the region of feature extractability.

5. The method of claim 4 wherein the contour line enclosing the region of feature extractability is manually drawn by a user over the displayed scanned image of the microarray using a touch screen device.

6. The method of claim 4 wherein the contour line enclosing the region of feature extractability is manually drawn by a user over the displayed scanned image of the microarray using a light pen.

7. The method of claim 4 wherein the contour line enclosing the region of feature extractability is manually drawn by a user over the displayed scanned image of the microarray using mouse and keyboard input.

8. The method of claim 1 wherein constructing a regularly shaped region of feature extractability from the received boundary of a region of feature extractability within the microarray further includes:
employing nearest neighbor analysis of pixels within the region of feature extractability to generate a binary mask containing binary values, each binary value indicating whether or not a corresponding pixel belongs to a feature-extractable region; and
determining a regularly shaped region of region of feature extractability from the binary mask.

9. The method of claim 8 wherein employing nearest neighbor analysis of pixels within the region of feature extractability to generate a binary mask further includes:
for each pixel,
sorting intensity values of nearest neighbor pixels to the pixel;
computing the average intensity of the nearest neighbor pixels;
when more than a threshold number of nearest neighbor intensity values are greater than the computed average intensity, setting a binary value in the binary mask corresponding to the pixel to indicate that the pixel is in a region of feature extractability; and
when a threshold number or less than a threshold number of nearest neighbor intensity values are greater than the computed average intensity, setting a binary value in the binary mask corresponding to the pixel to indicate that the pixel is not in a region of feature extractability.

10. The method of claim 8 wherein determining a regularly shaped region of region of feature extractability from the binary mask further includes:
computing a size of a regularly shaped region of feature extractability based on the binary mask; and
positioning the regularly shaped region of feature extractability so that the geometric center of the regularly shaped region of feature extractability coincides with a center of mass computed for the binary mask.

11. The method of claim 8 wherein computing a size of a regularly shaped region of feature extractability based on the binary mask further includes:
determining a size of a regularly shaped region of feature extractability so that a majority of pixels with corresponding binary-mask values indicating that the pixels are in a region of feature extractability are included in the regularly shaped region of feature extractability.

12. The method of claim 11 wherein the regularly shaped region of feature extractability includes one of:
a rectangular region specified by the lengths of two sides;
a disk-shaped region specified by a radius; and
an ellipsoid region specified by a major and a minor axis.

13. The method of claim 1 further comprising forwarding, to a remote location, feature-signal data extracted from the regularly shaped region of feature extractability.

14. A computer program implementing the method of claim 1 stored in a computer-readable medium.

15. Feature-signal data extracted from the regularly shaped region of feature extractability, determined by the method of claim 1, stored in a computer readable medium.

16. A microarray data processing system comprising:
a processor;
stored, computer readable microarray data;
a display device and a user input device; and
a program that
renders the microarray data for visual display;
displays the microarray data rendered for visual display;
receives a boundary of a region of feature extractability within the microarray; and
constructs a regularly shaped region of feature extractability from the received boundary of the region of feature extractability within the microarray.

17. The microarray data processing system of claim 16 wherein the program renders the microarray data for visual display by preparing a pixel-based, scanned image of the microarray with indications of putative feature positions.

18. The microarray data processing system of claim 17 wherein the program displays the microarray data rendered for visual display by displaying, on a computer display device, the pixel-based, scanned image of the microarray with indications of putative feature positions.

19. The microarray data processing system of claim 16 wherein the program receives a boundary of a region of feature extractability within the microarray by receiving a contour line enclosing the region of feature extractability.

20. The microarray data processing system of claim 16 wherein the program constructs a regularly shaped region of feature extractability from the received boundary of a region of feature extractability within the microarray by:
employing nearest neighbor analysis of pixels within the region of feature extractability to generate a binary mask with binary values, each binary value indicating whether or not a corresponding pixel belongs to a feature-extractable region; and
determining a regularly shaped region of region of feature extractability from the binary mask.

21. The microarray data processing system of claim 20 wherein the program employs nearest neighbor analysis of pixels within the region of feature extractability to generate a binary mask by:
for each pixel,
sorting intensity values of nearest neighbor pixels to the pixel;
computing the average intensity of the nearest neighbor pixels;
when more than a threshold number of nearest neighbor intensity values are greater than the computed average intensity, setting a binary value in the binary mask corresponding to the pixel to indicate that the pixel is in a region of feature extractability; and
when a threshold number or less than a threshold number of nearest neighbor intensity values are greater than the computed average intensity, setting a binary value in the binary mask corresponding to the pixel to indicate that the pixel is not in a region of feature extractability.

22. The microarray data processing system of claim 20 wherein the program determines a regularly shaped region of region of feature extractability from the binary mask by:
computing a size of a regularly shaped region of feature extractability based on the binary mask; and
positioning the regularly shaped region of feature extractability so that the geometric center of the regularly shaped region of feature extractability coincides with a center of mass computed for the binary mask.

23. The microarray data processing system of claim 22 wherein the program computes a size of a regularly shaped region of feature extractability based on the binary mask by:
determining a size of a regularly shaped region of feature extractability so that a majority of pixels with corresponding binary-mask values indicating that the pixels are in a region of feature extractability are included in the regularly shaped region of feature extractability.

24. The microarray data processing system of claim 16 wherein the regularly shaped region of feature extractability is one of:
a rectangular region specified by the lengths of two sides;
a disk-shaped region specified by a radius; and
an ellipsoid region specified by a major and a minor axis.

25. A method for processing microarray data, the method comprising:
rendering the microarray data for visual display;
displaying the microarray data rendered for visual display;
receiving as input an irregularly shaped region of feature extractability within the microarray;
constructing a regularly shaped region of feature extractability from the received boundary of the region of feature extractability within the microarray; and
extracting feature signals from the regularly shaped region of feature extractability.

26. A microarray data processing system comprising:
a processor;
stored, computer readable microarray data;
a display device and a user input device; and
a program that
renders the microarray data for visual display;
displays the microarray data rendered for visual display;
receives as input an irregularly shaped region of feature extractability within the microarray; and
constructs a regularly shaped region of feature extractability from the received boundary of the region of feature extractability within the microarray.
